(19) United States
(12) Patent Application Publication
(10) Pub. No.: US 2025/0339702 A1
(43) Pub. Date: Nov. 6, 2025

(54) Devices, Systems, and Methods for Application of Radio Frequency Energy to Sub-Cutaneous Tissue

(71) Applicant: BiLLite Medical LLC, Shreveport, LA (US)

(72) Inventors: Gerard Dayle Henry, Shreveport, LA (US); Robert A. Van Wyk, St. Petersburg, FL (US); LeRoy A. Jones, Austin, TX (US)

(21) Appl. No.: 19/193,789

(22) Filed: Apr. 29, 2025

Related U.S. Application Data
(60) Provisional application No. 63/641,876, filed on May 2, 2024.

(51) Int. Cl. A61N 1/40; A61N 1/05

(52) U.S. Cl. CPC A61N 1/403; A61N 1/0504; A61B 2018/00464; A61B 2090/395

(57) ABSTRACT

Radio frequency energy treatment is provided to subcutaneous tissue. Risk factors are reduced by employing one or more features. Electrodes that are inserted through the skin may be insulated except near the distal tips and/or the distance where the energy is provided may be at least at a minimum depth set by the treatment tool. The tissue may be treated with a power level that only denatures the tissue. The site may be analyzed with ultrasound to develop a treatment plan. A template may be provided to allow marking of the site where the skin should be pierced. A separate piercing tool may be used, thereby allowing the electrodes to have non-sharp shapes. A temperature sensor may be included to monitor temperature and/or impedance monitoring may be done where either or both may contribute to the control of RF energy and/or provide an alarm.

### FIELD OF THE INVENTION

Embodiments disclosed herein relate generally to the field of electrosurgery, and more particularly, to electrosurgical devices and methods which use radio frequency (RF) energy to cut, ablate, denaturize, coagulate and treat soft tissue lesions. The electrosurgical devices of the instant invention find particular utility in percutaneous thermal treatment of subcutaneous tissues of various types.

### BACKGROUND

Currently, for medical issues of the subcutaneous tissue, treatments are either external to the skin and are mostly weak and ineffective or the other extreme of may require surgical excision with significant anesthesia requirements. Medical issues of the subcutaneous layer have been limited to surgical excision (including, for instance, excision of a lipoma, open fat removal or liposuction) or application of transdermal pads (“coolsculpting”) which can be weak and ineffective for treating this layer of the body.

Surgical excision and liposuction treatments usually require systemic anesthesia (MAC or General) with the associated inherent risks. This is in addition to the morbidity of surgical excision including bleeding, infection, injury to nearby tissue injury, pain issues, and poor healing. As stated previously, external treatments are largely ineffective for treating subcutaneous tissue conditions as they must have energy transfer directly through the skin.

There is a need for improved devices and methods for the treatment of subcutaneous tissue with reduced risk factors.

### Basic Skin Anatomy:

Referring to FIG. 1, skin 900 is composed of 3 layers: the epidermis 902 is the outermost layer and is composed of keratinocytes or skin cells that form the “bricks” of the skin's barrier. The functions of the epidermis are protection from environmental insults (like ultraviolet light and toxins), prevention of dryness, and immune surveillance. The base of the epidermis is called the basal layer 904—it contains the cells that replicate to replace the epidermis every month. Beneath the epidermis is the dermis 906 composed mostly of collagen but also adjunctive structures like hair follicles and sweat glands. The dermis also contains vital blood vessels and nerves which traverse the collagen network there. The function of the dermis is temperature regulation through the secretion of sweat to the skin's surface and the regulation of blood flow to the area. Below the dermis, lies the subcutis [or hypodermis] 908 which holds fat and blood vessels. Fat is arranged into lobules that are several millimeters wide. The subcutis 908 acts as a heat insulator and provides protection from mechanical trauma.

Currently in conventional methods and systems, the technology for RF treatment for the skin is given above or at the level of the epidermis 902 and dermis 904 as depicted in FIGS. 2A and 2B. In FIG. 2A electrodes 910 are applied to the skin where the energy can cause skin injury, thermal damage, poor healing of the skin and be painful to the patient. In FIG. 2A electrodes 910 connected to RF generator 916 are traversed across epidermis 902 and dermis 904 to create elongate thermally treated zone 914 spanning the length 911, in the process creating regions 912 in which tissue is necrosed by extreme local temperatures and vaporization. In FIG. 2B a stationary array of electrodes 918 is connected to RF generator 916. Flow of RF energy between electrodes 918 creates region 914 in which tissue has been subjected to energy densities and resulting temperatures to achieve a desired effect. Regions of epidermis 902 and dermis 904 in direct contact with, and close proximity to electrodes 918 will be subjected to much higher temperatures than tissue in zone 914 likely resulting in tissue necrosis. Therefore, it is apparent that such conventional methods and systems of applying RF technology for treatment of the skin are unsatisfactory for treatment of subcutaneous tissue 908.

### SUMMARY

Embodiments disclosed herein address issues such as these and others by providing various features that allow for RF treatment of the subcutaneous tissue. Various embodiments may include one or more features that reduce one or more of the risk factors typically associated with such RF treatment.

Embodiments provide a method for treating subcutaneous tissue that includes a method of treating subcutaneous tissue. The method involves performing an ultrasound analysis at a subcutaneous depth over an area to be treated to develop a treatment plan. The metho further involves inserting at least one electrode of a radio frequency tool into the subcutaneous tissue at the area to be treated and applying radio frequency energy from the at least one electrode and into the subcutaneous tissue to provide treatment to the subcutaneous tissue.

Embodiments provide a system for treating subcutaneous tissue that includes a template having perforations separate by a first distance and configured to receive a marker through the perforations for marking skin of a patient. The system further includes a radio frequency tool having first and second electrodes separated by the first distance and a radio frequency generator electrically coupled to the first and second electrodes.

Embodiments provide a method of treating subcutaneous tissue that involves placing a template on skin of a patient over an area to be treated, the template including perforations spaced apart at a first distance. The method involves marking the skin through the perforations and creating openings through the skin at the markings. The method also involves passing first and second electrodes that extend from a radio frequency tool and that are separated by the first distance through the openings and into the subcutaneous tissue. The method further involves applying radio frequency energy from the first and second electrodes and into the subcutaneous tissue to provide treatment to the subcutaneous tissue.

Embodiments provide a system for treating subcutaneous tissue that includes a piercing tool having at least one sharpened element configured to pierce skin of the patient. The system includes a radio frequency tool having at least one electrode configured to reach subcutaneous tissue by passing through the pierced skin and to provide radio frequency energy to the subcutaneous tissue. The system includes a radio frequency generator electrically coupled to the at least one electrode.

Embodiments provide a method of treating subcutaneous tissue that involves piercing skin of a patent above a subcutaneous area to create at least one opening through the skin. The method involves passing at least one non-sharpened electrode that extends from a radio frequency tool through the at least one opening through the skin and into the subcutaneous tissue. The method also involves applying radio frequency energy from the at least one electrode into the subcutaneous tissue to provide treatment to the subcutaneous tissue.

Embodiments provide a system for treating subcutaneous tissue that includes a radio frequency tool having a first electrode and a second electrode configured to reach subcutaneous tissue and to provide radio frequency energy to the subcutaneous tissue. The first electrode and the second electrode have unsharpened distal tips with a shape that is distinct from proximal regions of the first and second electrodes. The system includes a radio frequency generator electrically coupled to the first and second electrodes.

Embodiments provide a method of treating subcutaneous tissue that involves passing a first electrode and a second electrode that extends from a radio frequency tool through openings through the skin and into the subcutaneous tissue. Unsharpened distal tips of the first and second electrodes may have a different shape than proximal portions of the first and second electrodes. The method further involves applying radio frequency energy from the distal tips of the first and second electrodes into the subcutaneous tissue to provide treatment to the subcutaneous tissue.

Embodiments provide a system for treating subcutaneous tissue that includes a radio frequency tool having at least one electrode that is configured to reach subcutaneous tissue and to provide radio frequency energy to the subcutaneous tissue. The at least one electrode has a proximal portion and a distal tip wherein the proximal portion is insulated while the distal tip is not insulated. The system further includes a radio frequency generator electrically coupled to the at least one electrode.

Embodiments provide a method of treating subcutaneous tissue that involves passing at least one electrode that extends from a radio frequency tool through an opening through the skin and into the subcutaneous tissue. The at least one electrode has a proximal portion and a distal tip wherein the proximal portion is insulated while the distal tip is not insulated. The method further involves applying radio frequency energy from the distal tips of the first and second electrodes into the subcutaneous tissue to provide treatment to the subcutaneous tissue.

Embodiments provide a system for treating subcutaneous tissue that includes a radio frequency tool having at least one electrode that is configured to reach subcutaneous tissue and to provide radio frequency energy at less than 25 Watts to the subcutaneous tissue. The system further includes a radio frequency generator electrically coupled to the at least one electrode and configured to produce less than 25 Watts of radio frequency energy.

Embodiments provide a method of treating subcutaneous tissue that involves passing at least one electrode that extends from a radio frequency tool through an opening through the skin and into the subcutaneous tissue. The method further involves applying radio frequency energy at less than 25 Watts from the distal tips of the first and second electrodes into the subcutaneous tissue to provide treatment to the subcutaneous tissue.

Embodiments provide a system for treating subcutaneous tissue that includes a radio frequency tool having a first electrode and a second electrode that are configured to reach subcutaneous tissue and to provide radio frequency energy to the subcutaneous tissue. The system further includes a radio frequency generator electrically coupled to the first electrode and the second electrode and configured to monitor an impedance occurring within the subcutaneous tissue between the first electrode and the second electrode in order to control the radio frequency energy and/or to provide an alarm based on the impedance.

Embodiments provide a method of treating subcutaneous tissue that involves passing a first electrode and a second electrode that extend from a radio frequency tool through an opening through the skin and into the subcutaneous tissue. The method further involves applying radio frequency energy from the distal tips of the first and second electrodes into the subcutaneous tissue to provide treatment to the subcutaneous tissue while monitoring an impedance of the subcutaneous tissue between the first electrode and the second electrode. The method also involves controlling the radio frequency energy and/or providing an alarm based on the impedance.

Embodiments provide a system for treating subcutaneous tissue that includes a radio frequency tool having at least one electrode that is configured to reach subcutaneous tissue and to provide radio frequency energy to the subcutaneous tissue. The radio frequency tool further includes a housing having a distal end that the at least one electrode extends from and having a temperature sensor on the distal end that is configured to contact skin of a patient. The system also includes a radio frequency generator electrically coupled to the at least one electrode and configured to monitor a temperature from the temperature sensor in order to control the radio frequency energy and/or to provide an alarm based on the temperature.

Embodiments provide a method of treating subcutaneous tissue that involves passing at least one electrode that extends from a radio frequency tool through an opening through the skin and into the subcutaneous tissue, where the radio frequency tool includes a housing having a distal end that the at least one electrode extends from and having a temperature sensor on the distal end that is configured to contact skin of a patient. The method also involves applying radio frequency energy from a distal tip of the at least one electrode into the subcutaneous tissue to provide treatment to the subcutaneous tissue while monitoring a temperature from the temperature sensor. The method further involves controlling the radio frequency energy and/or providing an alarm based on the temperature.

Embodiments provide a system for treating subcutaneous tissue that includes a radio frequency tool having at least one electrode that is configured to reach subcutaneous tissue and to provide radio frequency energy to the subcutaneous tissue, where the at least one electrode has a distal tip having a temperature sensor. The system further includes a radio frequency generator electrically coupled to the at least one electrode and configured to monitor a temperature from the temperature sensor in order to control the radio frequency energy and/or to provide an alarm based on the temperature.

Embodiments provide a method of treating subcutaneous tissue that involves passing at least one electrode that extends from a radio frequency tool through an opening through the skin and into the subcutaneous tissue, where the at least one electrode has a distal tip having a temperature sensor. The method involves applying radio frequency energy from a distal tip of the at least one electrode into the subcutaneous tissue to provide treatment to the subcutaneous tissue while monitoring a temperature from the temperature sensor. The method further involves controlling the radio frequency energy and/or providing an alarm based on the temperature.

Embodiments provide a system for treating subcutaneous tissue that includes a radio frequency tool having at least one electrode that is configured to reach subcutaneous tissue and to provide radio frequency energy to the subcutaneous tissue. The system further includes a radio frequency generator electrically coupled to the at least one electrode and configured to control the radio frequency energy to denature the subcutaneous tissue surrounding the at least one electrode.

Embodiments provide a method of treating subcutaneous tissue that involves passing at least one electrode that extends from a radio frequency tool through an opening through the skin and into the subcutaneous tissue. The method further involves applying radio frequency energy from a distal tip of the at least one electrode into the subcutaneous tissue to denature the subcutaneous tissue.

### DESCRIPTION OF THE DRAWINGS

FIG. 1 is a diagrammatic depiction of skin anatomy and structures.

FIG. 2A depicts a first prior art method for RF treatment for the skin given above or at the level of the epidermis/dermis.

FIG. 2B depicts a second prior art method for RF treatment for the skin given above or at the level of the epidermis/dermis.

FIG. 3 depicts a pair of bipolar electrodes inserted through the dermis into the subcutaneous layer/hypodermal space.

FIG. 4 depicts the flow of RF energy through tissue and fluids between a bipolar electrode pair.

FIG. 5A depicts the energy flow between two needle electrodes in a homogeneous liquid or other conductive medium when bipolar energy is supplied safely below the dermis.

FIG. 5B depicts the energy flow of FIG. 5A when the energy is too close to the dermis.

FIG. 6 depicts a small (less than 7 mm) vein at the completion of treatment.

FIG. 7 is a distal perspective exploded view of a percutaneous thermal treatment device of the present invention.

FIG. 8 is a proximal perspective exploded view of the objects of FIG. 7.

FIG. 9 is a plan view of a bipolar device of the present invention for the subcutaneous thermal treatment of tissue, with its axially positionable bipolar electrodes in a first position and the positioning latch in its locked position.

FIG. 10 is a side elevational view of the objects of FIG. 9.

FIG. 11 is a bottom plan view of the objects of FIG. 9.

FIG. 12 is a proximal upper perspective view of the objects of FIG. 9.

FIG. 13 is a lower distal perspective view of the objects of FIG. 9.

FIG. 14 is an expanded view of the objects of FIG. 9 at location C.

FIG. 15 is an expanded view of the objects of FIG. 13 at location A.

FIG. 16 is an expanded view of the objects of FIG. 9 at location D.

FIG. 17 is a side elevational view of the device of FIG. 9 with the positioning latch in its unlocked position in preparation for repositioning of the bipolar electrodes.

FIG. 18 is a lower perspective view of the objects of FIG. 16.

FIG. 19 is a plan view of the device of FIG. 9 with the positionable electrodes in their fully retracted position.

FIG. 20 is a side elevational view of the objects of FIG. 18.

FIG. 21 is an expanded perspective view of the distal portion of the objects of FIG. 18.

FIG. 22 is a plan view of the device of FIG. 9 with the position able electrodes in their fully extended position.

FIG. 23 is a side elevational view of the objects of FIG. 21.

FIG. 24 is an expanded perspective view of the distal portion of the objects of FIG. 21.

FIG. 25 is a perspective view of elements of a percutaneous thermal treatment system of the present invention.

FIG. 26 is a plan view of a template of the present invention for use with the device of FIG. 9.

FIG. 27 is a side elevational view of the objects of FIG. 24.

FIG. 28 is a perspective view of the objects of FIG. 24.

FIG. 29 is a perspective view of a piercing device of the present invention for use with the device of FIG. 9.

FIG. 30 is a plan view of the objects of FIG. 27.

FIG. 31 is a side elevational view of the objects of FIG. 27.

FIG. 32 depicts an ultrasound device in use in a first step in the subdermal thermal treatment of tissue using devices and methods of the present invention.

FIG. 33 depicts the template of FIG. 4 in use in a second step in the subdermal thermal treatment of tissue using devices and methods of the present invention.

FIG. 34 depicts positioning of the piercing device of FIG. 27 in preparation for use in a third step in the subdermal thermal treatment of tissue using devices and methods of the present invention.

FIG. 35 depicts the piercing device of FIG. 27 in use in a fourth step in the subdermal thermal treatment of tissue using devices and methods of the present invention.

FIG. 36 depicts the bipolar device of FIG. 9 positioned for use in a subsequent step of subdermal thermal treatment of tissue using devices and methods of the present invention.

FIG. 37 depicts the objects of FIG. 34 during activation showing the flow of RF energy through tissue undergoing thermal treatment using devices and methods of the present invention showing the flow of RF energy through targeted tissue.

FIG. 38 depicts an alternate embodiment of the presentation in use as in FIG. 37.

FIG. 39 depicts the distal portion of an alternate embodiment thermal treatment device of the present invention.

FIG. 40 is an expanded sectional view of the objects of FIG. 39 at location C-C.

FIG. 41 is a distal elevational view of the distal portion of an alternate embodiment thermal treatment device of the present invention.

FIG. 42 is a side elevational view of the objects of FIG. 41.

FIG. 43 is an expanded sectional view of the objects of FIG. 42 at location B-B.

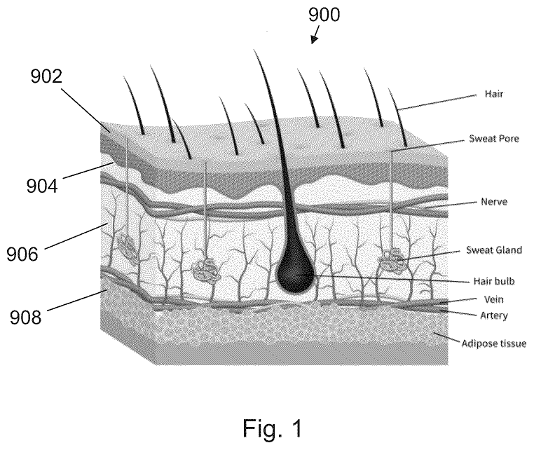

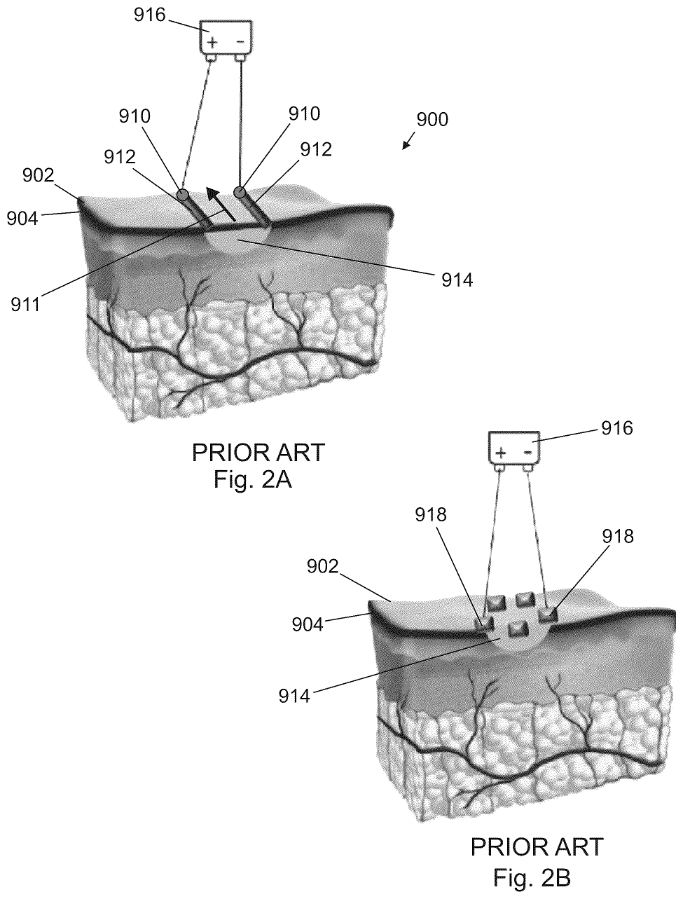

### DETAILED DESCRIPTION

Embodiments disclosed herein in the field of electrosurgery, more particularly, to high efficiency electrosurgical surgical instruments and methods which use radio frequency (RF) electrical power to percutaneously denature, desiccate, coagulate and ablate subcutaneous soft tissues.

Specific embodiments of devices and methods are discussed in more detail below. However, before these devices and methods are described in further detail, it is to be understood that the detailed description is not intended to be limiting, while embodiments that may successfully provide the treatment are not limited to the particular compositions, methodologies or protocols herein described, as these may vary in accordance with routine experimentation and optimization. It is also to be understood that the terminology used in the description is for the purpose of describing the particular versions or embodiments only and is not intended to limit the scope of the present invention.

In the context of this detailed description of the various embodiments, the following definitions apply:

The words “a,” “an,” and “the” as used herein mean “at least one” unless otherwise specifically indicated.

The present invention makes reference to an “electrode.” As used herein, the term “electrode” refers to one or more conductive elements formed from any suitable metallic material, such as stainless steel, nickel, titanium, tungsten, and the like, connected, for example via cabling disposed within the elongated proximal portion of the instrument, to a power supply, for example, an externally disposed electrosurgical generator, and capable of generating an electric field.

The term “proximal” refers to that end or portion which is situated closest to the user; in other words, the proximal end of an electrosurgical device of the instant invention will typically include the handle portion.

The term “distal” refers to that end or portion situated farthest away from the user; in other words, the distal end of an electrosurgical instrument of the instant invention will typically include the bipolar electrode portions.

The present invention makes reference to the thermal treatment of tissue. As used herein, the term “tissue” refers to biological tissues, generally defined as a collection of interconnected cells that perform a similar function within an organism. Embodiments are not limited in terms of the tissue types to be treated but rather may have broad application to the thermal treatment of any target tissue with particular applicability to the ablation, denaturation, or desiccation of subcutaneous tissue.

The term “denature” or “denaturation” as used herein refers to the causation of cell lysis, without breakdown of the bonds between cells, minimal liquefaction and no charring. Cell membranes could be intact but internal components are disrupted. Denatured tissue is absorbed by the body after treatment.

As used herein the term “ablation” refers to non-destructive thermal treatment of tissue using RF energy for the purpose of denaturation or desiccation.

The embodiments disclosed herein may have both human medical and veterinary applications. Accordingly, the terms “subject” and “patient” are used interchangeably herein to refer to the person or animal being treated or examined. Exemplary animals include house pets, farm animals, and zoo animals, especially mammals.

Unless otherwise defined, all technical and scientific terms used herein have the same meaning as commonly understood by one of ordinary skill in the art to which this invention belongs. In case of conflict, the present specification, including definitions, will control.

One or more of the embodiments of methods and devices disclosed herein affect tissue below the epidermis and dermis in the subcutaneous layer while reducing or eliminating thermal effects at the epidermis and dermis. Bipolar RF energy is applied in the subcutis to thermally treat larger adipose and smaller vessels thereby largely avoiding the epidermis/dermis complex where thermal injury typically occurs that can result in poor wound healing and pain. The one or more embodiments disclosed herein target these larger adipose/small vessels of the sub-cutaneous layer. For example, lipoma's [benign tumor made of fat tissue that grows under the skin] currently require open surgical excision.

Major concerns when performing cutaneous RF treatment are worries about thermal injury, pain and wound healing. One or more embodiments disclosed herein may incorporate a pair of parallel, distally extending electrode elements that are separated by a fixed distance. In one or more embodiments, the proximal portions of these electrode elements are insulated so that only the uninsulated distal ends of the elements can conduct RF energy to tissue and fluids in contact therewith. Because of this, thermal effects are largely limited to regions well below the skin and undesirable dermal affects are avoided. The electrode elements are positioned parallel to each other, and the uninsulated portions are positioned more than a cm below the skin surface before the energy is applied to the sub-cutaneous substance—also known as hypodermis.

Additionally, in one or more embodiments disclosed herein, the RF generator monitors the tissue impedance and has an impedance alarm enabling the energy level to be kept less than that required to produce dermal injury but enough energy to treat the lesion of interest. In some embodiments the hand piece incorporates a thermal sensor that monitors skin temperature so as to enable the system to shut off energy if skin temperature rises. The thermal sensor is located in a distal-facing surface that is pressed against the skin during treatment thereby ensuring that the electrode elements are fully inserted to a predetermined treatment depth that prevents superficial activation and resulting dermal injury.

In embodiments of methods of thermal treatment disclosed herein, ultrasound imaging is used to examine skin and subsurface tissue to ensure that no significant large vessels, fluid collections or other abnormalities are present subcutaneously. This ultrasound examination will ensure adequate subcutaneous fat depth of at least an adequate amount for the particular patient in the treated area.

In some embodiments, thermal treatment systems of the present invention operate at low energy levels, preferably twenty-four Watts or less which is very low compared to current treatment regimens. In some embodiments wherein an RF generator monitors impedance between the electrodes, the RF generator may use algorithms to detect the completion of treatment by changes in the impedance and alert the clinician or terminate activation.

While electrosurgery is commonly used for cutting, coagulating and ablating tissue, one or more embodiments disclosed herein may be used to denature the subcutaneous tissue and have the body to naturally absorb the denatured tissue. This is particularly advantageous as it provides clinicians with the ability to concentrate on specific areas of subcutaneous tissue that patients have been unable to lose/reduce with standard weight loss management. Surgical excision of these small, localized fat deposits results in risk outweighing benefits, for reasons stated already.

When using various embodiments disclosed herein, only topical local anesthesia may be required. This eliminates risks and costs associated with general anesthesia.

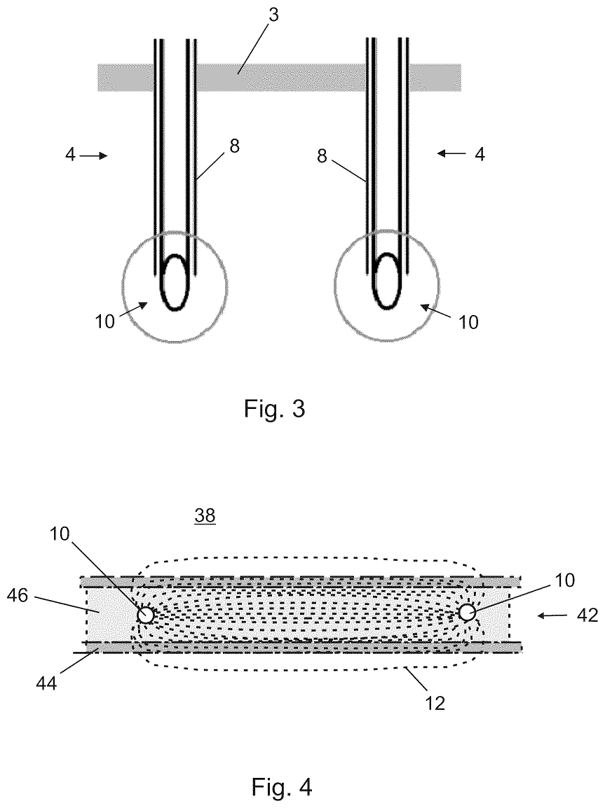

While monopolar electrosurgical devices use a single electrode tip which delivers energy to tissue in the conductive path between the electrode and the grounding plate, bipolar devices of the present invention apply RF energy between two electrodes positioned under the skin to avoid injury to the skin/dermal complex. Proximal portions of the electrodes that contact the epidermis and dermis are insulated while distal portions positioned in the subcutaneous adipose tissue are uninsulated so that RF energy passes through the tissue between the uninsulated portions. By avoiding the flow of RF energy through epidermis and dermal layers of the skin, devices of the present invention prevent thermal damage to the skin. Because of this, and due to the minimally invasive nature of embodiments of the methods and systems disclosed herein, injury to the skin is minimal and less patient pain results (see FIG. 3). With bipolar RF, the energy current runs from the active to the return electrode through the tissue and conductive fluids mainly between the two electrodes. (see FIG. 4). As a result of avoiding the application of RF energy to the epidermis and dermis layers of the skin, the patient experiences less direct heating of these tissues resulting in less thermal injury and better post operative healing of the area. Unlike current RF therapy to the skin, where the energy first and lastly goes through the epidermis, devices and methods of the present invention avoid not only the epidermis, but the dermis as well.

One or more embodiments of systems and methods for subcutaneous thermal tissue treatment may incorporate additional devices that together may be supplied to the clinician as a kit. These include a template for marking insertion locations for the treatment electrodes, and a piercing device used to produce perforations in the skin for insertion of the treatment electrodes. When treating a patient using embodiments of methods and systems disclosed herein, the region to be treated may first be examined using ultrasound imaging to identify locations for electrode insertion. Thereafter a template is used to mark the insertion locations using a suitable skin marker. In addition, the patient will be able to see exactly where the treatment is to be given prior to proceeding ahead.

Some embodiments of the methods and systems may include a piercing device that has a pair of sharpened protruding distal elements spaced the same distance apart as the electrodes on the RF treatment device to be used. Sharpened distal elements of the piercing device are inserted into the skin so as to create perforations for insertion of the treatment electrodes. The piercing elements are inserted to a depth sufficient to allow insertion of the electrodes to the treatment location. The piercing device is removed, and electrodes of the treatment device are fully inserted until a distal face of the handpiece is pressed against the skin so that a thermal sensor is in firm contact with the skin. The power level for treatment may be selected by the clinician or the electrosurgical generator may establish it automatically based on safety to the measured impedance level and algorithms within the generator. Thereafter the generator is activated, and RF energy is applied to the site until impedance detected by the generator increases to a predetermined safety value whereupon activation is terminated. The electrodes are then withdrawn from the site. During activation, the skin temperature is monitored via the thermal sensor on the device which is connected by wires to the generator. If the skin temperature exceeds a preset safety value during treatment, the supply of RF energy to the handpiece may be suspended until the temperature falls to an acceptable preset value.

The heating effect of RF energy is proportional to the density of the energy flow through the tissue. The flow of RF energy from a monopolar electrode to a remotely located return electrode is essentially omnidirectional. The energy density and therefore the rate of heating at a location in proximity to the electrode is primarily determined by its distance from the electrode and the applied power level. Accordingly, determining when a given thermal effect has been achieved for a given tissue mass is relatively straightforward. When thermally treating tissue with bipolar devices with the electrodes mounted at a distance from one another, the energy flow is strongly affected by the location of the electrodes, particularly their distance one from another. Determining when treatment is complete at desired locations between the electrodes using impedance is difficult since the heating is dependent on variables including the electrode spacing and the exposed (conductive) area of the electrodes in contact with tissue.

Bipolar devices of the present invention have distally extending electrodes that are inserted percutaneously at the location of tissue to be treated, the electrodes being at a fixed distance one from another, and axially movable to establish a fixed distance to which the electrodes may be inserted. Skin and subcutaneous tissue proximal to the treatment site through which the electrodes are inserted is protected by insulating sleeves covering the proximal portions of the electrodes adjacent to the handle of the device.

Accordingly, when thermally treating tissue using one or more embodiments of devices and systems disclosed herein, the tissue type, electrode spacing, electrode depth and exposed electrode areas are all known prior to treatment. In some embodiments these variables are supplied to the electrosurgical generator wherein optimal characteristics for the RF output are calculated using algorithms. In some embodiments the output of the generator is formed of pulses of RF energy. The “on time,” “off time,” and amplitude (power level) of this pulse train may be optimized for thermal treatment of the specified tissue in a manner that allows complete treatment without damage to surrounding tissue. In some embodiments the generator monitors the impedance between the electrodes and based on the impedance value, determines when treatment is complete.

The rate of temperature rise of tissue at a location through which RF energy passes is proportional to the density of the energy through that location. Energy density and therefore temperature are highest in close proximity to an electrode from which RF energy flows. This can lead to desiccation of tissue near the electrode. Desiccated tissue has a high impedance and limits the flow of energy from an electrode. If the temperature of tissue adjacent to or in contact with the electrode exceeds 100 C liquid from the tissue can boil causing arcing between the electrode and tissue, which in turn may cause the formation of char on the electrode. The desiccated tissue and/or char, in turn, insulates the electrode and prevents or severely diminishes subsequent flow of RF energy. This may prevent successful treatment of tissue at the site. Therefore, one or more embodiments disclosed herein may prevent overheating of the electrodes used for thermal treatment of tissue. The one or more embodiments may achieve this by several methods, either singly or in combination. Current flow and energy density are concentrated at corners and sharp points. Accordingly, in some embodiments the electrode distal ends are hemispherical to prevent energy concentration. In other embodiments the electrodes are each an assembly with a sharpened distal end, the distal end being formed of a ceramic material. In yet other embodiments each electrode has a central lumen terminating near the distal end of the electrode. Thermocouples positioned in these lumens are connected to the generator wherein circuitry monitors the electrode temperatures and adaptively controls the power output to prevent overheating of the electrodes. The benefits of these adaptive methods may be enhanced by constructing the electrodes from metallic materials of high thermal conductivity. Biocompatibility coatings may be applied to high conductivity materials that would not otherwise be suitably compatible for patient exposure. Accordingly, these one or more embodiments can prevent the formation of char or excessive desiccation of tissue in contact with, or close proximity to the electrode.

Some embodiments of bipolar devices may operate at low RF energy levels of 25 watts or less, to avoid thermal injury and char. In some embodiments an impedance alarm is set to a level at which the subcutaneous tissue is only denatured, and liquification and/or boiling is precluded. The “on time,” “off time,” and amplitude (power level) of the RF pulse train may be optimized for thermal treatment of the specified tissue in a manner that allows adequate treatment without injury to surrounding tissue. In some embodiments the generator monitors the impedance between the electrodes and based on the impedance value, determines when treatment is complete. Ultrasound imaging is used to ensure adequate depth of the subcutaneous tissue prior to treatment and evaluate tissue being treated. In some embodiments of the methods disclosed herein, a piercing device and marking of the skin are used to ensure desired location of treatment to the doctor and the patient. In some embodiments of the devices disclosed herein, a temperature probe is used to ensure that the distal surface of the device handle is against the skin during treatment and functions as a safety energy shutoff if the skin temperature rises above a predetermined value.

FIGS. 3 through 5 depict the bipolar distal tips 10 for percutaneous thermal tissue treatment. Referring now to FIG. 3, the distal tips 10 are not insulated and can conduct energy whereas the insulated 8 parts of the entire needle complex 4 travels through the skin 3 into the subcutaneous layer. Therefore, negligible energy is being applied to the regions of tissue adjacent the insulated parts 8. The insulation for the insulated parts 8 may be of various types. External surfaces of insulated portion 8 may be covered with an insulative dielectric coating, such as polymeric heat-shrink tubing formed of, for instance, polyolefin, polyvinylidene fluoride (PVDF), or polytetrafluoroethylene (PTFE), among others.

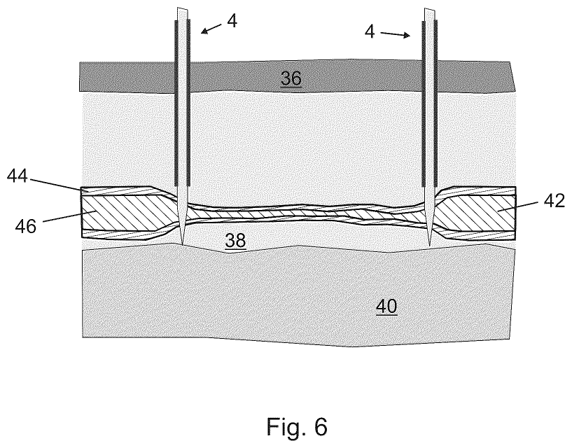

FIG. 4 depicts (in a sectional plan view, looking down on the site) treatment of a small vessel 42 in the subcutis surrounded by fatty tissue 38 wherein distal ends 10 of electrodes of the needle complex 4 are inserted directly into vessel 42. The impedance (resistance) of wall 44 of vessel 42, and fluid filling lumen 46 of vessel 42 are both low, while the impedance of surrounding fatty tissue 38 is high. As a result, energy flow 12 is concentrated in vessel 42 while flow through surrounding fatty tissue 38 is minimal. Surrounding fatty tissue 38 is also a poor thermal conductor. As a result, vessel 42 experiences heating caused by RF energy flowing through vessel 42 resulting in denaturing of vessel wall 44 and shrinkage of collagen forming wall 44. At completion of treatment, vessel 42 appears as depicted in FIG. 6.

In FIGS. 4 and 6 distal ends 10 of electrode 4 are in direct contact with vessel 42, and indeed penetrate wall 44 so that RF energy is applied directly to vessel 42. The low impedance of vessel 42 compared to fatty tissue 38 with which vessel 42 is surrounded will cause current to preferentially flow through vessel 42 even when electrode distal ends 10 are not in direct contact with vessel 42 but only in proximity. As a result, small vessels in fatty tissue undergoing treatment have higher energy density flow than the surrounding tissue and be affected more quickly than the surrounding fatty tissue. This effect will rapidly decrease as the vessel tissue becomes denatured and desiccated.

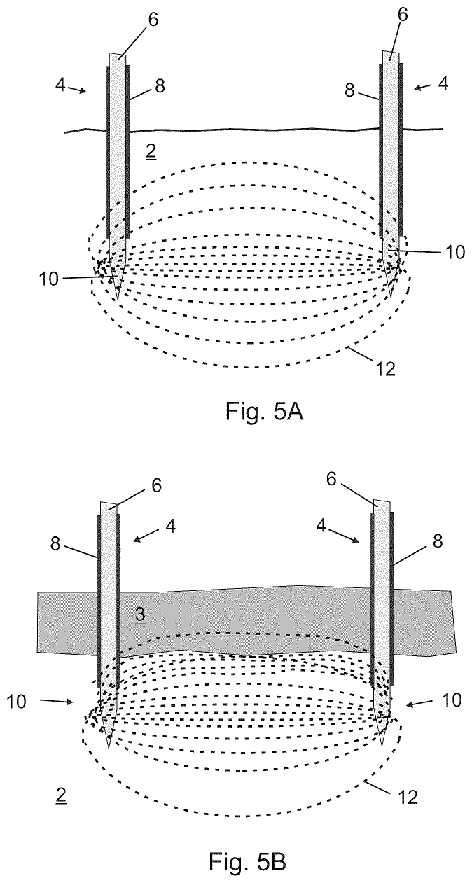

FIG. 5A depicts the flow of energy between needle electrodes 6 in tissue wherein bipolar energy is supplied with uninsulated portions 10 of electrodes 6 safely below the dermis. The flow of energy 12 is symmetrically concentrated primarily in the shortest path between uninsulated portions 10, and decreases with increasing distance away from this shortest path. By keeping uninsulated portions 10 sufficient distance from the dermis the flow of energy through the dermis is minimized so as to prevent heating of the dermis.

FIG. 5B depicts the energy flow 12 wherein uninsulated portions 10 are not inserted to sufficient depth to minimize the flow of energy 12 through dermis 3. Insufficient insertion depth may result in thermal damage to dermis 3.

Referring again to FIG. 6 depicting vessel 42 after treatment with bipolar RF in the subcutaneous layer 38 below the skin 36 and above deep tissue/muscle 40, portions of vessel 42 not between electrode tips 10 will be unaffected by the energy flow given from the needle complex assemblies 4 after treatment is completed.

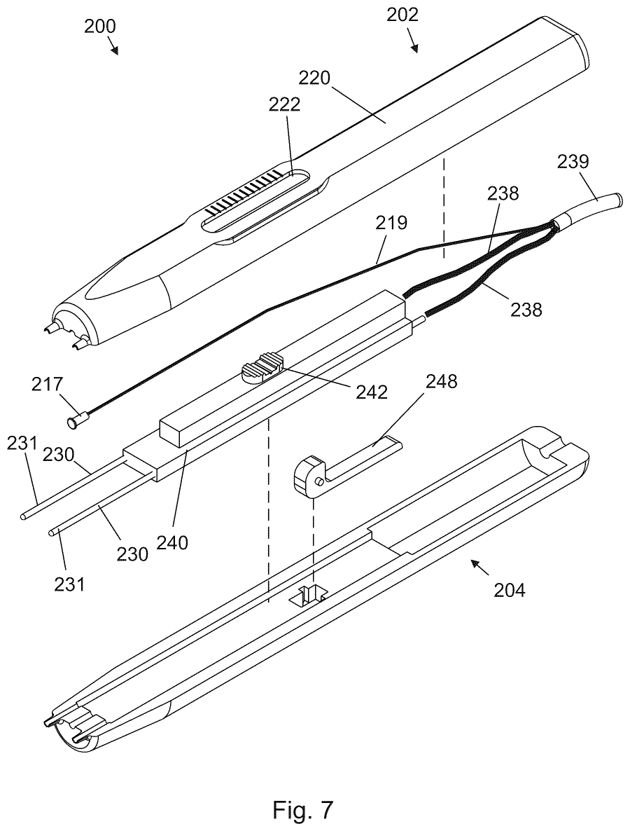

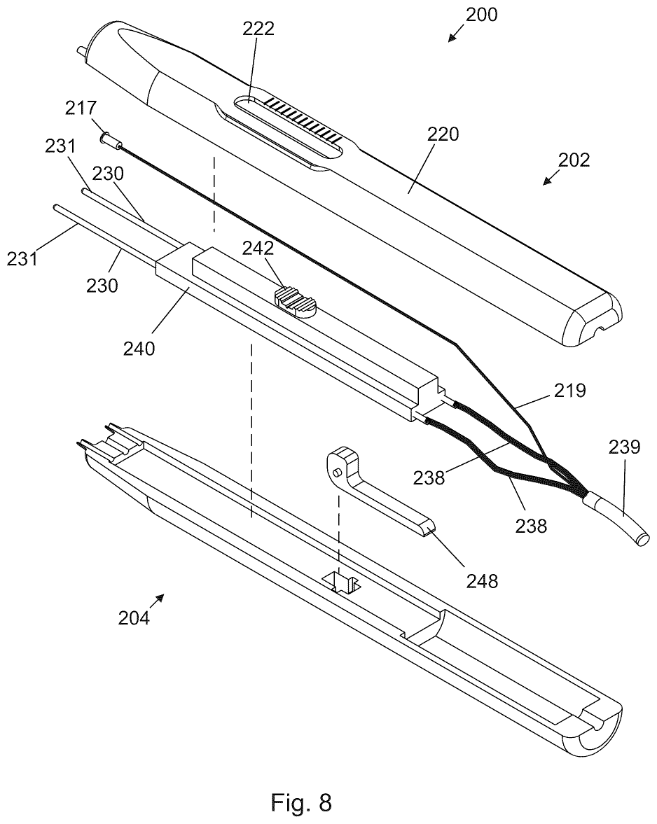

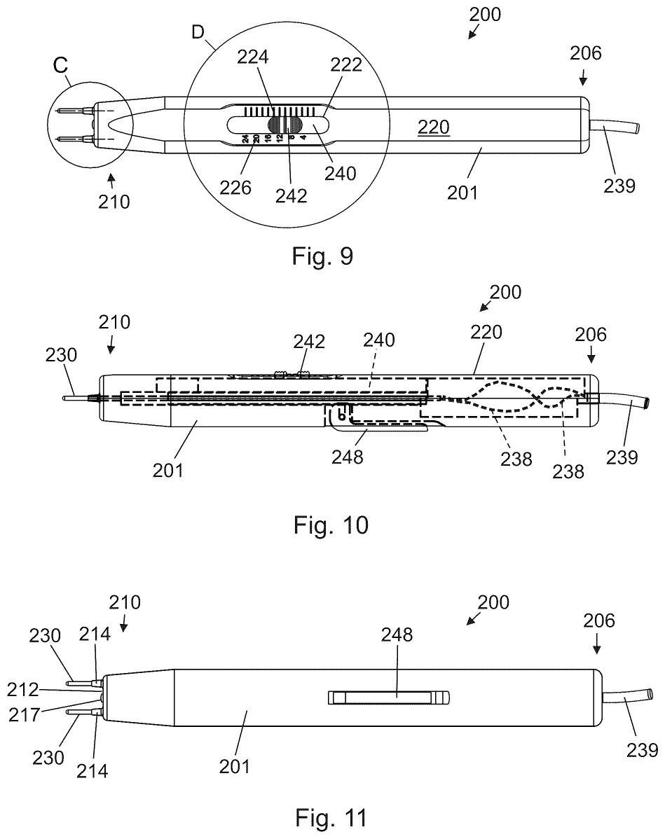

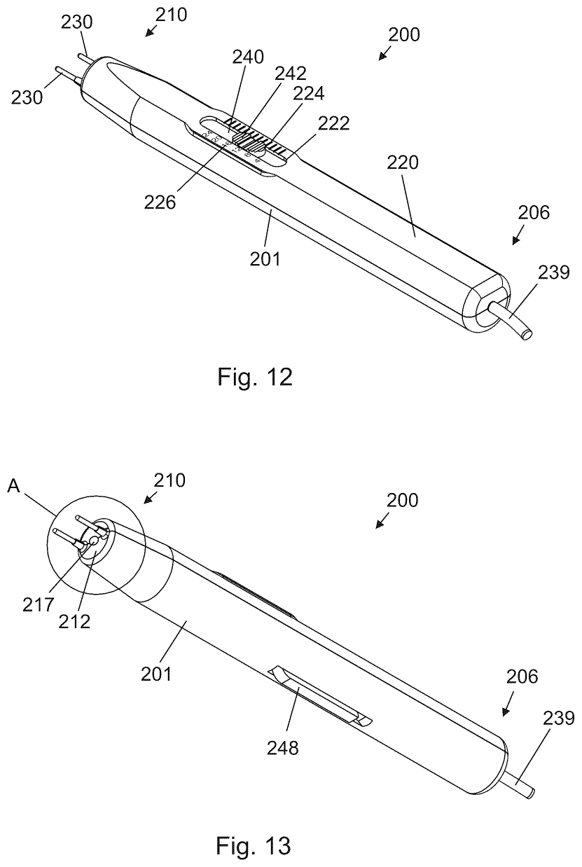

FIGS. 7 through 16 depict one example of a bipolar device 200 for percutaneous thermal tissue treatment. Referring now to FIGS. 7 and 8, device 200 has a molded polymeric handle housing 201, as indicated at FIG. 9, formed by top half 202 and bottom half 204 wherein is slidably positioned slide 240 with upwardly protruding control portion 242. Distal portions 231 of electrodes 230 protrude distally from slide 240 to which they are affixed. Proximal portions of electrodes 230 are connected by wires 238 to cable 239. Wires 238 have sufficient length to allow unimpeded motion of slide 240 and electrodes 230 mounted thereto. Top half 202 of handle housing 201 has a top surface 220 in which is formed elongate opening 222, positioned such that control portion 242 of slide 240 protrudes therethrough when handle 201 is assembled as depicted in FIGS. 9 through 16. Slide positioning lock 248 is rotatably positioned in lower half 204 prior to assembly to upper half 202.

Additionally, the bipolar device 200 may include a temperature sensor 217 such as a thermocouple that is connected to one or more signal conductors 219. The temperature sensor 217 is positioned on the distal end so that when the electrodes 230 are inserted to the subcutaneous tissue, the temperature sensor 217 contacts the skin of the patient and can monitor the temperature of the skin during the procedure, thereby allowing the temperature of the skin to control the radio frequency energy being provided, including causing the radio frequency to shut off, and/or causing an audible and/or visible alarm to be provided by a radio frequency generated that is connected to the wires 219, 238.

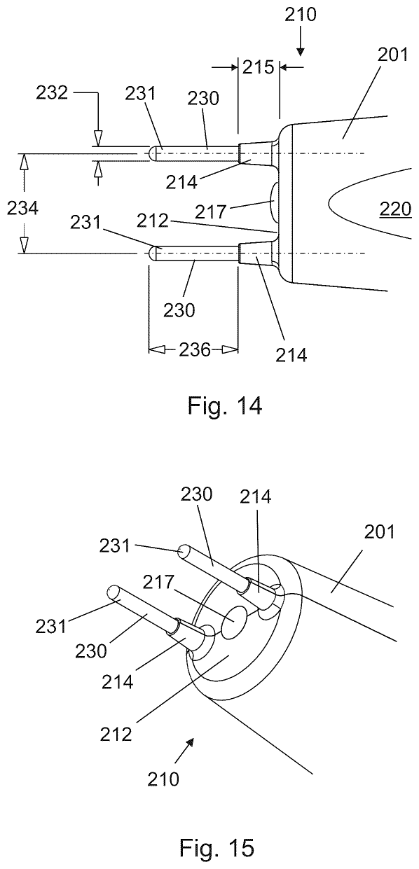

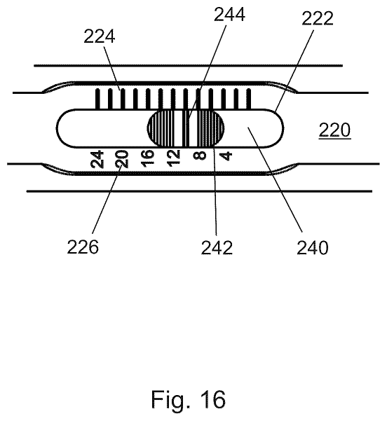

Referring now to FIGS. 9 through 16, handle 201 has a proximal end 206 from which passes cable 239, and a distal end 210 with distal-most surface 212 on which are positioned tubular portions 214 from which protrude distal portions 231 of electrodes 230 slidably positioned therein. As best seen in FIG. 14, electrodes 230 of diameter 232 are positioned distance 234 apart and protrude distance 236 beyond the distal limit of tubular protrusions 214. Referring to FIG. 16, gradations 224 and indicia 226 formed on surface 220 of handle 221 adjacent to slot 222 together with indicating feature 244 on control portion 242 of slide 240 indicate the protrusion distance 236 of distal portions 231 of electrodes 230. In some embodiments distal ends of electrodes 230 are rounded. In other embodiments the distal ends are sharp, capable of piercing tissue. As depicted in FIGS. 9 through 16 the axial position of slide 240 and electrodes 230 mounted thereto is fixed, positioning lock 248 being in its first locked position.

While size and proportions of various aspects of the embodiments may vary from one example to another, in some embodiments diameter 232 is between 0.5 and 5.0 millimeters. In some embodiments diameter 232 is between one millimeter and 4 millimeters. In still other embodiments electrodes 230, or at least the uninsulated portion, may have a non-cylindrical cross-section that may be elliptical, oblong, or a combination of circular, linear and curvilinear segments. For instance, the uninsulated portions may be paddle shaped. In such cases, the insulated proximal portions may remain cylindrical while the uninsulated portions such as the distal tips may be of a different shape than the proximal portions.

In some embodiments height 215 of cylindrical protrusions 214 is 20 millimeters or less. In some embodiments height 215 is 15 millimeters or less. Height 215 is selected by the surgeon to suit the treatment requirements. Again, such dimensions may vary from one example to another depending upon the application.

In some embodiments distance 234 between electrodes 230 is between two millimeters and 30 millimeters. In some embodiments distance 234 is between 3 and 20 millimeters. Distance 234 is selected by the surgeon to suit the treatment requirements. Again, such dimensions may vary from one example to another depending upon the application.

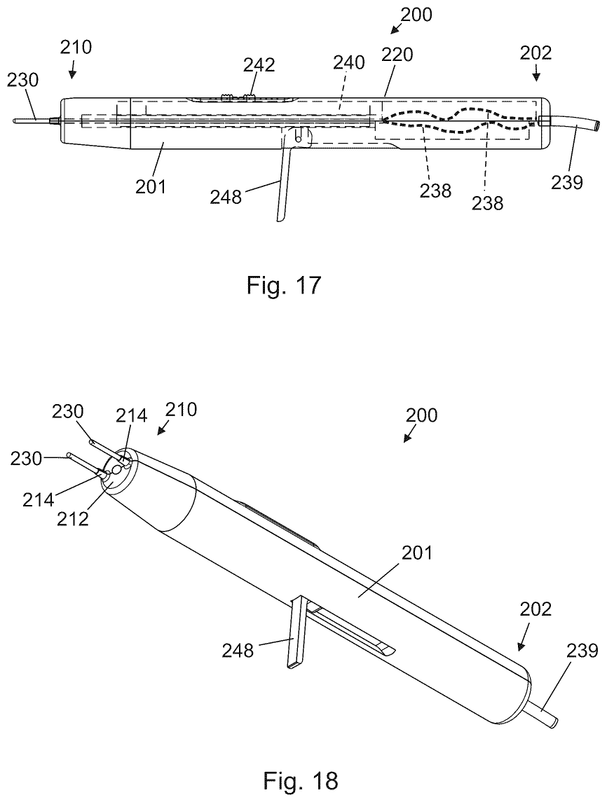

FIGS. 17 and 18 depict device 200 with positioning lock 248 being rotated into its second condition in which the position of slide 240 is not fixed. Protrusion distance 236 of distal portions 231 of electrodes 230 may be set to a pre-selected value using control portion 242 of slide 240 to position slide 240, the distance being indicated by feature 244, graduations 244 and indicia 246. When the desired distance 236 is indicated, positioning lock 248 is returned to its first, locked position and movement of slide 240 is precluded.

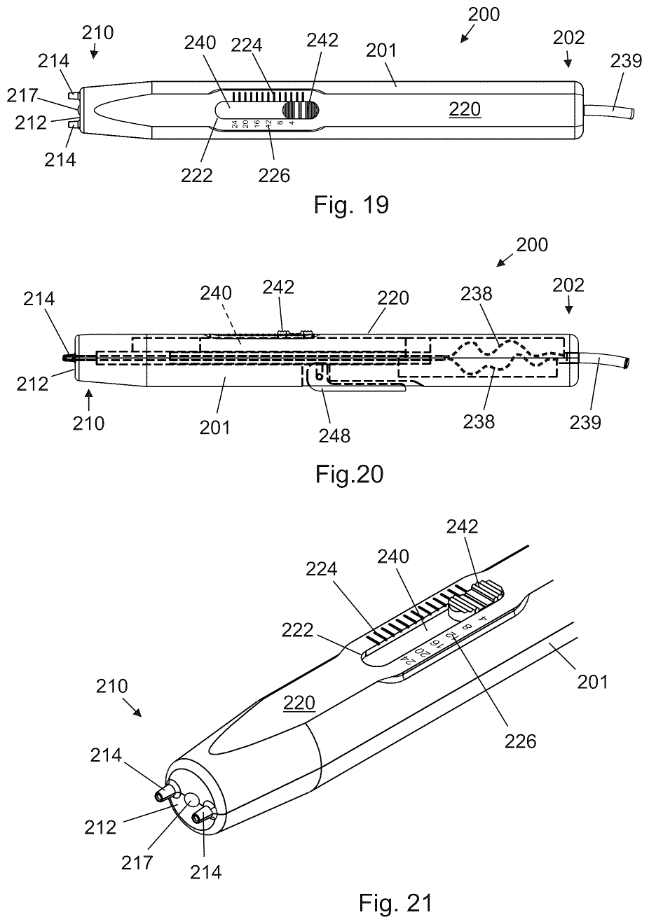

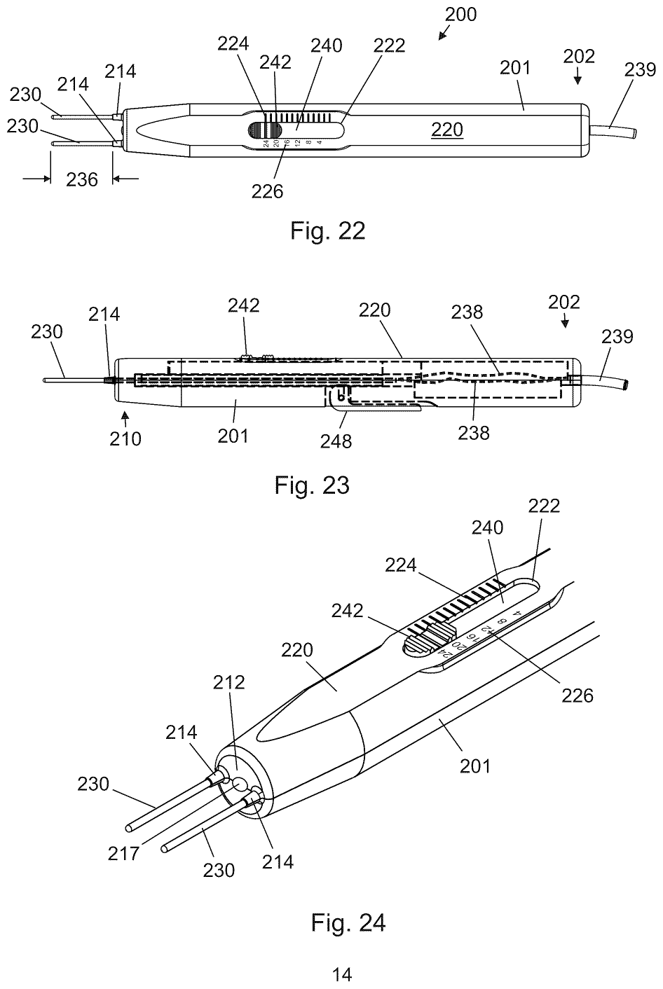

FIGS. 19 through 21 depict device 200 with electrodes 230 in their fully retracted, safe position as indicated by feature 244 of control portion 242 of slide 240 and gradations 224 and indicia 226. Slide lock 248 is in its first, locked position. In FIGS. 22 through 24 electrodes 230 are extended to their maximum distance 236. In some embodiments maximum distance 236 is 40 millimeters. In some embodiments maximum distance 236 is 30 millimeters. Again, such dimensions may vary from one example to another depending upon the application.

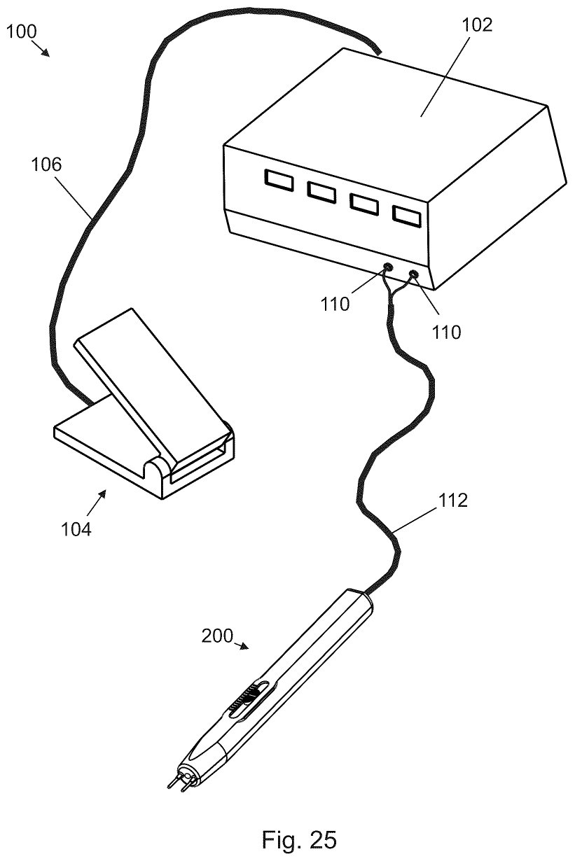

FIG. 25 depicts a system 100 of the current invention for percutaneous thermal treatment of tissue. Generator 102 is connected to foot pedal 104 by cable 106. Device 200 is connected to bipolar outputs 110 of generator 102 by cable 112.

Generator 102 is configured to provide RF energy in the form of pulses with the pulses being separated by sufficient time to allow electrodes 230 to cool sufficiently to prevent the formation of char on the distal portions of electrodes 108. The pulse frequency may vary from example to example depending upon the application, but in some embodiments ranges of from about 450 kilohertz to about 2 megahertz. Between pulses of RF energy, circuitry within generator 102 measures the impedance between electrodes 230 which allows control of the radio frequency energy based on the impedance, including shutting off the treatment and/or generating an audible and/or visible alarm. For instance, the generator 102 may shut off therapy and/or produce an alarm upon detecting that the impedance indicates the subcutaneous tissue being treated has become denatured. Impedance above a predetermined value indicates maximal treatment of subcutaneous tissue such as a vein. The distance 234 between electrodes 230 strongly affects the measured impedance of a vein segment, longer distances creating proportionately greater impedance between electrodes 230. As noted above, in one or more of these embodiments treatment may be terminated when the impedance increases to a predetermined fixed value. In other embodiments, an initial impedance value between electrodes 230 is determined prior to the initiation. Treatment is terminated when the impedance between electrodes 230 reaches a predetermined value based on the initial impedance value. In some embodiments electrode characteristics such as exposed area, spacing and tissue type are supplied to generator 102, and generator 102 determines optimal output characteristics and the impedance level for determining completion of treatment.

The effect of RF energy applied to a tissue is strongly affected by its electrical and thermal conductivity. Electrical conductivity (in Siemens/meter) and thermal conductivity (Watts/meter/degree Celsius) for tissue types are given below.

| Tissue | Electrical | Thermal |
|---|---|---|
| Blood | .66 | .52 |
| Blood vessel wall | .23 | .46 |
| Extracellular fluid | 2.7 | .60 |
| Fat | .08 | .21 |
| Muscle | .46 | .49 |
| Skin | .15 | .37 |

Blood is very conductive electrically and thermally. Blood vessel walls are less electrically conductive than blood itself but still have fairly high thermal conductivity. This fat which has very low electrical conductivity and very low thermal conductivity. When thermally treating a blood vessel surrounded by fat, the flow of RF energy is as depicted in FIG. 4. The power output of a radiofrequency generator is determined by the circuitry within the generator in response to the impedance (resistance) that the generator senses between its leads. The relationship between output power level and measured impedance value is referred to as the generator's “load curve.” The impedance between a pair of bipolar electrodes is determined by the electrical conductivity of the tissue in the conductive path, the distance between the electrodes, and the electrode area in contact with the tissue. In some embodiments, the distance between the electrodes and exposed area of the electrodes are established by a handpiece. With these values fixed, the generator is able to determine the conductivity of the tissue and select a mode optimal for the tissue to be treated. The load curves/modes for treating highly conductive tissues like blood vessels will be very different from those for treating, for instance, fat with its low electrical and thermal conductivity, though the treatment of both falls within the scope of various embodiments disclosed herein. Ultrasound imaging may be used prior to treatment to evaluate the tissue being treated.

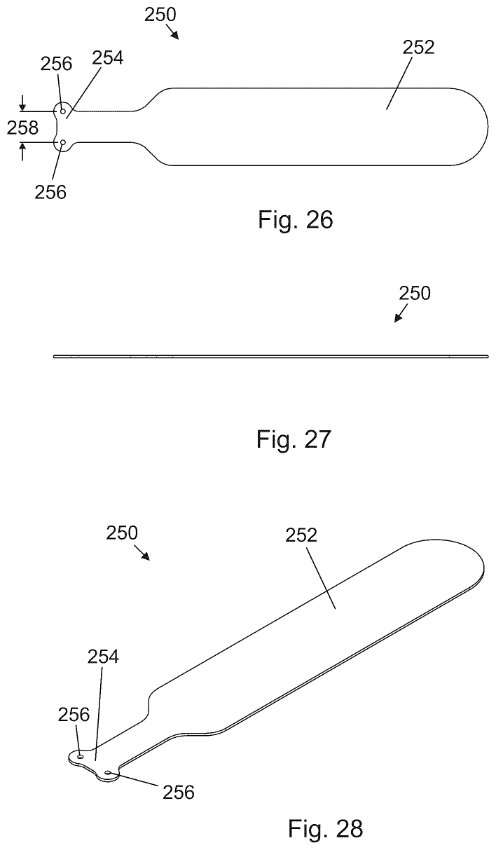

FIGS. 26 through 28 depict a template 250 that may be used in one or more embodiments of methods and systems for percutaneous thermal tissue treatment. Template 250, formed from a rigid thin sheet material, has a proximal handle portion has a proximal handle portion 252, and a distal portion 254 in which are formed perforations 256 spaced distance 258 apart. Distance 258 is equal to distance 234 between electrodes 230 (see FIG. 14). It will be appreciated that the template 250 may be used to mark the area prior to an ultrasound procedure so as to provide confirmation of the area of treatment to both the clinician and the patient. However, the template 250 may be used after the ultrasound procedure to mark the specific location where the electrodes should be inserted as discussed below.

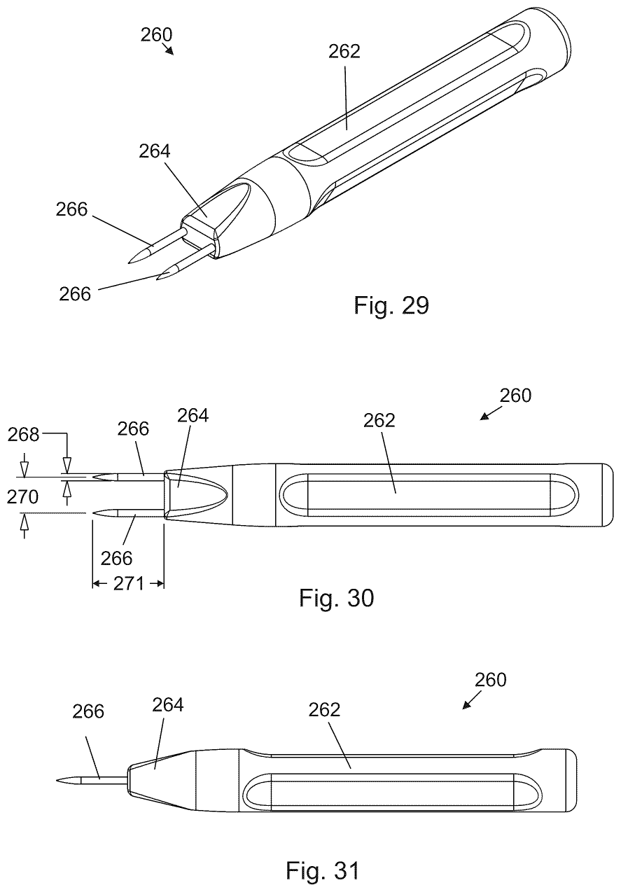

FIGS. 29 through 31 depict a piercing device that may be used in embodiments of methods and systems for percutaneous thermal tissue treatment. Piercing device 260 has a handle 262 with a distal end 264 from which protrude sharpened elements 266 of diameter 268 and length 271 spaced distance 270 apart. Distance 270 is equal to distance 234 between electrodes 230 (see FIG. 14). Diameter 268 is equal to, or greater than diameter 232 of electrodes 230.

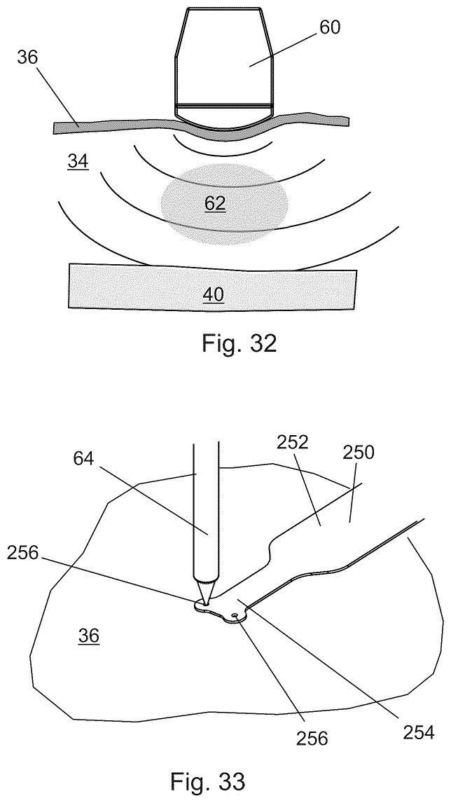

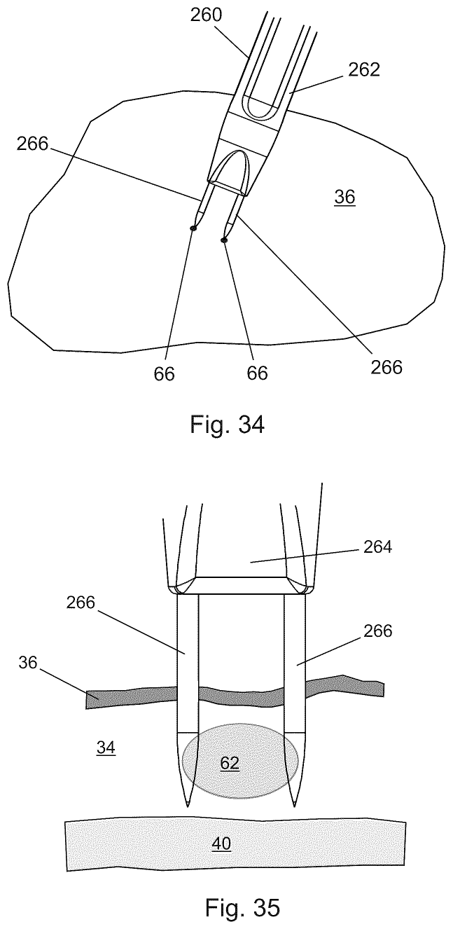

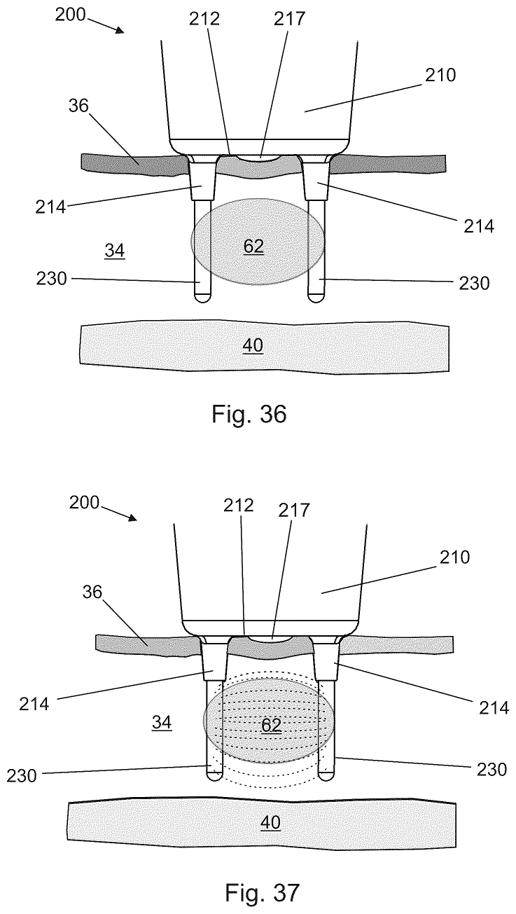

Hereafter is described are exemplary methods for thermally treating tissue subcutaneously using embodiments of devices disclosed herein. Referring now to FIG. 32, tissue 62 of interest is located in surrounding tissue 34 beneath skin 36 and above underlying tissue 40. The size and location of tissue 62 are evaluated using an ultrasound device 60. Thereafter, based on information garnered through the ultrasound, an RF thermal treatment plan is developed, the plan including the location and spacing of the electrodes and the placement depth of the electrodes. A template 250 with perforations 256 spaced the selected distance 258 apart is used as depicted in FIG. 33 to mark the locations for electrode insertion using tissue marker 64. The distal ends of 266 of piercing device 260 are positioned as shown in FIG. 34 using location marks 66 applied previously using template 250, and inserted through skin 36 to a predetermined depth as depicted in FIG. 35. Thereafter piercing device 260 is removed from the site. Using methods previously herein described, protrusion distance 236 of electrodes 230 of device 200 is set to a predetermined value and locked. Using perforations formed in skin 36 by piercing device 260, electrodes 230 are inserted and positioned as depicted in FIG. 36, conical protrusions 214 of device 200 forming an insulating barrier to prevent the flow of RF energy directly from electrodes 230 to skin 36 as depicted in FIG. 37.

FIG. 37 depicts the flow of RF energy through tissue 62, raising the temperature of tissue 62 so as to denature tissue 62. Subsequently, over time, tissue 62 will necrose and be absorbed by the body.

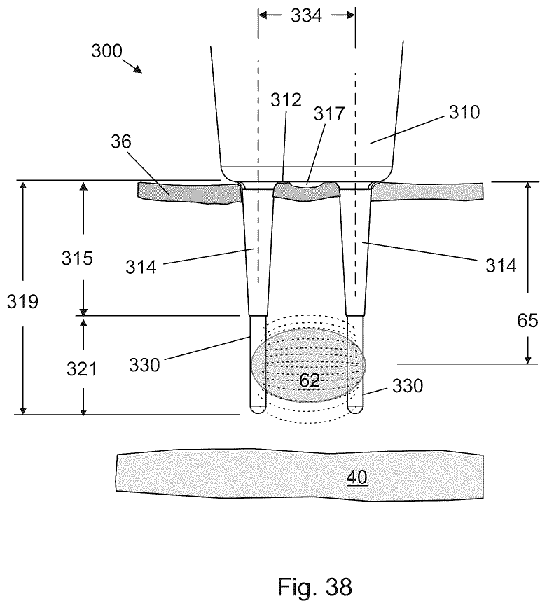

In FIG. 38 device 200 has been replaced by device 300 that is identical in all aspects of form and function to device 200 except as subsequently described. Distance 334 between electrodes 330 is reduced and height 315 of tubular protrusions 314 has been increased. Tissue 62 is reduced in size and at an increased depth 65 beneath skin 36. The changes are made to allow device 300 to treat tissue 62 at the increased distance 65 below skin 36 without affecting skin 36 and surrounding tissue proximal to electrodes 330. Distance 334 between electrodes 330 and their length 319, length 315 of tubular portions 314 and the resulting length of uninsulated portions of electrodes 330 are determined by the size 62 of the tissue to be treated, and the depth 65 of tissue 62 beneath the surface of dermis 36. Length 321 of conductive portions of electrodes 330 must be sufficient to ensure energy flow through the entirety of tissue 62. For small tissue masses, length 317 may be as little as one millimeter, however, decreasing the exposed conductive area increases the energy density at the electrode thereby increasing the local tissue temperature commensurately. As previously described herein, this can result in desiccation of tissue in contact with or immediately adjacent to the electrode, or the forming of char on the electrode. Either condition will prevent successful thermal treatment.

In FIG. 38 it can also be seen that the temperature sensor 317 such as a thermocouple is present on the distal end 310. The temperature sensor 317 is in contact with the skin 36 to monitor the temperature of the skin 36 during the treatment.

The proximal portions of electrodes 230 and 330 are insulated from surrounding tissue by tubular protrusions 214 and 314 that are part of the handle portions of respective devices 200 and 300. In other embodiments the proximal portions of the electrodes are insulated by dielectric coatings or other insulating elements (for instance, polymeric heat shrink tubing as previously discussed) applied to the electrodes. All fall within the scope of embodiments disclosed herein. So long as the distally extending electrodes have a proximal portion that is insulated or otherwise isolated from surrounding tissue in some manner, this protects the dermis and underlying tissues proximal to the treatment site.

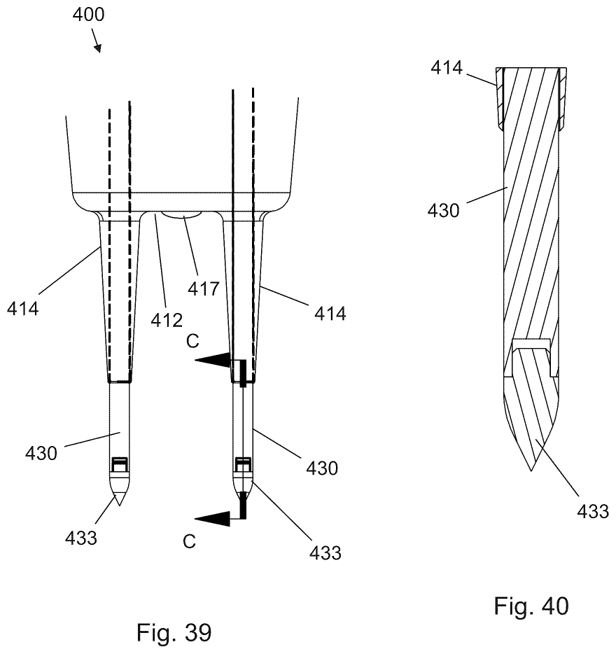

FIGS. 39 and 40 depict the distal portion of an alternate embodiment device 400 identical in all aspects of form and function to device 300 except as specifically described hereafter. Needle electrodes 430 of device 400 are not rounded like electrodes 330, but rather are an assembly with a sharpened ceramic element 433 affixed to their distal end. Ceramic tip 433 allows needle electrodes 430 to penetrate tissue thereby eliminating the need for piercing device 260. Energy flow will be restricted to the cylindrical surfaces of needle electrodes 430 thereby creating a more even heating effect than if the ends of electrodes 230 were simply sharpened. In FIG. 39 it can also be seen that the temperature sensor 417 such as a thermocouple is present on the distal end.

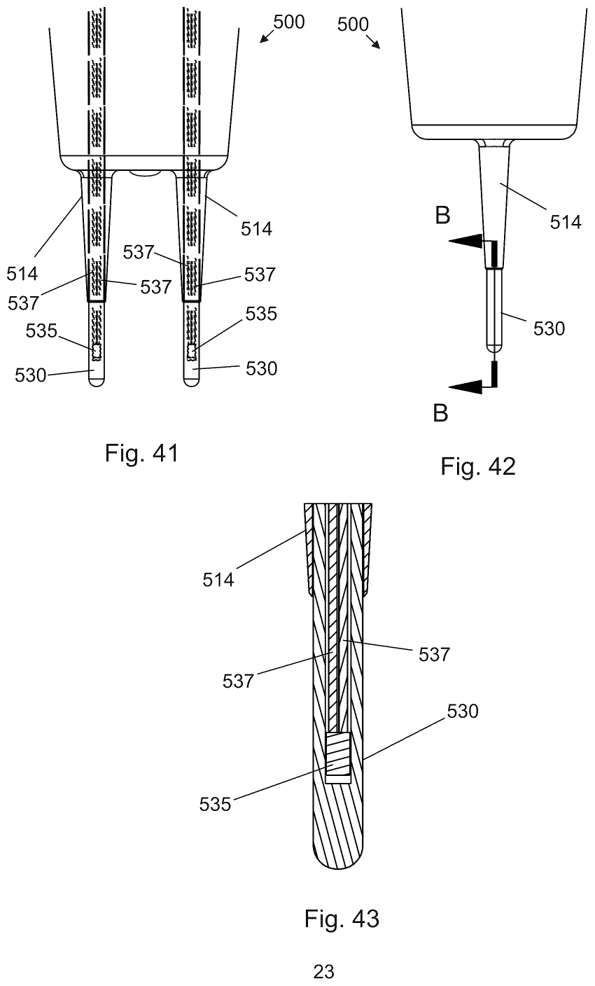

Alternative embodiment device 500 depicted in FIGS. 41 through 43 is like device 200 in all aspects of form and function except as specifically subsequently described. Electrodes 530 have formed therein a distally extending cannulation wherein is positioned a temperature sensor such as a thermocouple 535 connected to generator 102 by wires 537. Thermocouple provides real time temperature feedback from electrodes 530 to generator 102 so that the output power can be adjusted to keep the temperature of electrodes 530 within a predetermined range so as to prevent charring of tissue in contact with needles 530. Furthermore, the real time temperature feedback from the thermocouple 535 may trigger the generator 102 to cause the RF energy to shut off entirely and/or to produce an audible and/or visible alarm.

As can be appreciated from the description above, embodiments have provided various features that allow for RF energy treatment of subcutaneous tissue. Various risk factors have been addressed by the corresponding various features.

While embodiments have been particularly shown and described, it will be understood by those skilled in the art that various other changes in the form and details may be made therein without departing from the spirit and scope of the invention.

1. A method of treating subcutaneous tissue, comprising:
performing an ultrasound analysis at a subcutaneous depth over an area to be treated to develop a treatment plan;
inserting at least one electrode of a radio frequency tool into the subcutaneous tissue at the area to be treated; and
applying radio frequency energy from the at least one electrode and into the subcutaneous tissue to provide treatment to the subcutaneous tissue.

2. The method of claim 1, further comprising:
placing a template on skin of a patient over an area to be treated, the template including perforations spaced apart at a first distance;
marking the skin through the perforations;
creating openings through the skin at the markings;
passing first and second electrodes that extend from the radio frequency tool and that are separated by the first distance through the openings and into the subcutaneous tissue; and
applying radio frequency energy from the first and second electrodes and into the subcutaneous tissue to provide treatment to the subcutaneous tissue.

3. The method of claim 1, further comprising:
piercing skin of a patent above a subcutaneous area to create at least one opening through the skin, wherein the at least one electrode is non-sharpened.

4. The method of claim 1, further comprising:
passing a first electrode and a second electrode that extends from the radio frequency tool through openings through the skin and into the subcutaneous tissue, unsharpened distal tips of the first and second electrodes having a different shape than proximal portions of the first and second electrodes; and
applying radio frequency energy from the distal tips of the first and second electrodes into the subcutaneous tissue to provide treatment to the subcutaneous tissue.

5. The method of claim 1, wherein the at least one electrode has a proximal portion and a distal tip wherein the proximal portion is insulated while the distal tip is not insulated, the method further comprising applying radio frequency energy from the distal tips of the first and second electrodes into the subcutaneous tissue to provide treatment to the subcutaneous tissue.

6. The method of claim 1, further comprising:
applying radio frequency energy at less than 25 Watts from the distal tips of the first and second electrodes into the subcutaneous tissue to provide treatment to the subcutaneous tissue.

7. The method of claim 1, further comprising:
passing a first electrode and a second electrode that extend from a radio frequency tool through an opening through the skin and into the subcutaneous tissue;
applying radio frequency energy from the distal tips of the first and second electrodes into the subcutaneous tissue to provide treatment to the subcutaneous tissue while monitoring an impedance of the subcutaneous tissue between the first electrode and the second electrode; and
controlling the radio frequency energy and/or providing an alarm based on the impedance.

8. The method of claim 1, wherein the radio frequency tool comprises a housing having a distal end that the at least one electrode extends from and having a temperature sensor on the distal end that is configured to contact skin of a patient, the method further comprising:
applying radio frequency energy from a distal tip of the at least one electrode into the subcutaneous tissue to provide treatment to the subcutaneous tissue while monitoring a temperature from the temperature sensor; and
controlling the radio frequency energy and/or providing an alarm based on the temperature.

9. The method of claim 1, wherein the at least one electrode has a distal tip having a temperature sensor, the method further comprising:
applying radio frequency energy from a distal tip of the at least one electrode into the subcutaneous tissue to provide treatment to the subcutaneous tissue while monitoring a temperature from the temperature sensor; and
controlling the radio frequency energy and/or providing an alarm based on the temperature.

10. The method of claim 1, further comprising:
applying radio frequency energy from a distal tip of the at least one electrode into the subcutaneous tissue to denature the subcutaneous tissue.

11. A system for treating subcutaneous tissue, comprising:
a template having perforations separate by a first distance and configured to receive a marker through the perforations for marking skin of a patient;
a radio frequency tool having first and second electrodes separated by the first distance; and
a radio frequency generator electrically coupled to the first and second electrodes.

12. The system of claim 11, further comprising:
a piercing tool having at least one sharpened element to pierce skin of the patient, the first and second electrodes reach the subcutaneous tissue by passing through the pierced skin.

13. The system of claim 11, wherein the first electrode and the second electrode have unsharpened distal tips with a shape that is distinct from proximal regions of the first and second electrodes.

14. The system of claim 11, wherein the first electrode and the second electrode have a proximal portion and a distal tip wherein the proximal portion is insulated while the distal tip is not insulated.

15. The system of claim 11, wherein the radio frequency generator is configured to produce less than 25 Watts of radio frequency energy.

16. The system of claim 11, wherein the radio frequency generator is configured to monitor an impedance occurring within the subcutaneous tissue between the first electrode and the second electrode in order to control the radio frequency energy and/or to provide an alarm based on the impedance.

17. The system of claim 11, wherein the radio frequency tool further comprises a housing having a distal end that the first and second electrodes extend from and having a temperature sensor on the distal end that is configured to contact skin of a patient, and wherein the radio frequency generator is configured to monitor a temperature from the temperature sensor in order to control the radio frequency energy and/or to provide an alarm based on the temperature.

18. The system of claim 11, wherein at least one of the first and second electrodes has a distal tip having a temperature sensor, and wherein the radio frequency generator is configured to monitor a temperature from the temperature sensor in order to control the radio frequency energy and/or to provide an alarm based on the temperature.

19. The system of claim 11, wherein the radio frequency generator is configured to control the radio frequency energy to denature the subcutaneous tissue surrounding the at least one electrode.

20-99. (canceled)

100. A method of treating subcutaneous tissue, comprising:
passing at least one electrode that extends from a radio frequency tool through an opening through the skin and into the subcutaneous tissue; and
applying radio frequency energy from a distal tip of the at least one electrode into the subcutaneous tissue to denature the subcutaneous tissue.
